(19) United States
(12) Patent Application Publication
(10) Pub. No.: US 2023/0149072 A1
(43) Pub. Date: May 18, 2023

(54) DEVICE FOR OCCLUSION OF A LEFT ATRIAL APPENDAGE OF A HEART

(71) Applicant: AURIGEN MEDICAL LIMITED, Galway (IE)

(72) Inventors: Tony O'HALLORAN, Galway (IE); John THOMPSON, Dublin (IE); John KELLY, Galway (IE); Conor ALLEN, Galway (IE)

(73) Assignee: AURIGEN MEDICAL LIMITED, Galway (IE)

(21) Appl. No.: 17/995,840

(22) PCT Filed: Apr. 14, 2021
(86) PCT No.: PCT/EP2021/059715

§ 371 Date: Oct. 7, 2022

(30) Foreign Application Priority Data
Apr. 14, 2020 (GB) 2005439.1

(51) Int. Cl. A61B 18/14

(52) U.S. Cl. CPC A61B 18/1492; A61B 2018/00375

(57) ABSTRACT

A device for occlusion of a left atrial appendage of a heart including an implantable occlusion apparatus including a radially expansible element adjustable between a contracted orientation for transluminal delivery and a deployed orientation configured to occlude the left atrial appendage, an elongated catheter member operably and detachably attached to the implantable occlusion apparatus and configured for transluminal delivery and deployment of the occlusion apparatus in the left atrial appendage, and an energy delivery module configured for adjustment from a contracted configuration, and a deployed configuration suitable for engagement with adjacent tissue to ablate the tissue. The energy delivery module is operably attached to the elongated catheter member proximally of the radially expansible element and configured to contact and ablate a wall of the left atrium surrounding an ostium of the left atrial appendage.

### FIELD OF THE INVENTION

The present invention relates to a device for occlusion of a left atrial appendage of a heart, and methods for occlusion of the left appendage of the heart.

### BACKGROUND TO THE INVENTION

Atrial fibrillation (AF) is a common cardiac rhythm disorder affecting an estimated 6 million patients in the United States alone. AF is the second leading cause of stroke in the United States and may account for nearly one-third of strokes in the elderly. As our population continues to age, this problem may become even more prevalent. In greater than 90% of cases where a blood clot (thrombus) is found in the AF patient, the clot develops in the left atrial appendage (LAA) of the heart. The irregular heart beat in AF causes blood to pool in the left atrial appendage, because clotting occurs when blood is stagnant, clots or thrombi may form in the LAA. These blood clots may dislodge from the left atrial appendage and may enter the cranial circulation causing a stroke, the coronary circulation causing a myocardial infarction, the peripheral circulation causing limb ischemia, as well as other vascular beds. The LAA is a muscular pouch of heart attached to the left atrium. Mechanical occlusion of the LAA may result in a reduction of the incidence of stroke in AF patients, and there is growing interest in both surgical and endovascular methods to remove isolate the LAA.

New devices to percutaneously occlude the LAA have been developed for stroke prophylaxis and seem promising. These new devices include the use of a clip to clamp the LAA shut, the use of a snare to wall off the LAA, the use of an umbrella device to expand the LAA, the use of a device which may close the LAA but not obliterate it, and the use of a device which may fill the LAA without closing it. Data on the safety and efficacy of these devices must be considered over time. These new devices are early in clinical trials for human application and have several limitations. For instance, use of the clip to clamp the LAA shut may not get down to the base of the LAA, may leave a residual stump or leak, may result in a clot forming, and may require open surgery. Use of the snare may leave a residual stump or leak, may be less controlled, and may not be possible if adhesions are located around the heart. Use of the umbrella device may require the patient to be on blood thinners since it is made from a foreign material and does not occlude and obliterate the LAA simultaneously. Use of a device which may close the LAA without obliterating it, and use of a device which may obliterate the LAA without closing it are both incomplete solutions which may experience leakage, which may require blood thinners due to the use of synthetic materials, or which may experience other types of issues.

More recent devices proposed for occlusion of the LAA and prevention/treatment of atrial fibrillation and LAA-associated thrombotic events are described. WO2012/109297 describes an implantable device having an expandable LAA-occluding barrier and anchor configured for engagement of the ostium of the LAA, a pacing module for treatment of atrial fibrillation, and a sensor for detecting the electrical activity of the heart indicative of arrhythmia. WO2013/009872 describes a LAA-occluding device configured to inject a filler material into the LAA, and including a transponder unit configured to detect and relay to an external base station data electrical parameters of the LAA tissue. WO2016/202708 describes an implantable device having a LAA occluding body, electrodes configured to heat LAA tissue with a view to electrical isolation of the LAA, and sensors configured to determine heat or electrical activity of the LAA, which signals are used as feedback to control the heating of the tissue.

PCT/EP2018/058799 discloses a device for occlusion of the left atrial appendage (LAA) of the heart. The device comprises a radially expansible element (generally a cage) with an open distal end, a side wall and a concave proximal end. A connecting hub for detachable attachment to a delivery catheter is provided on the concave end. A fluid impermeable cover is disposed on the proximal end of the cage, proximal of the connecting hub, to prevent passage of fluid through the device. The cover has a re-closable aperture to allow access to the connecting hub while maintaining a fluid tight seal. The cage is configured for radial expansion from a contracted orientation suitable for transluminal delivery and a deployed radially expanded configuration suitable for occlusion of a body lumen such as the LAA. The device without the cover is shown in Figure A, and with the cover in Figure B. The device includes treatment electrodes configured to ablate tissue within the LAA, generally adjacent to the sidewall of the cage, to electrically isolate the LAA, and sensors configured to detect electrical parameters of the LAA tissue distal of the ablation zone.

US2020/000870 describes a device for ablation and occlusion of the left atrial appendage of the heart. The device comprises a catheter with a distal end, an expandable occlusive member disposed on the distal end of the catheter, and an energy delivery member disposed on the catheter proximal of the occlusive member. The energy delivery member comprises an inflatable balloon configured for radially outward inflation into contact with the ostium of the LAA, having electrodes disposed on an inner wall of the balloon. In use, the balloon is positioned inside the mouth of the LAA and inflated into contact with the ostium of the LAA, and ablative energy is delivered to the ostium by the electrodes (FIG. 7). A problem with this device is that the provision of electrodes on an inner surface of the balloon inhibits energy transfer to tissue on the opposite side of the balloon, making it difficult to treat the tissue of take impedance measurement of the adjacent tissue.

### SUMMARY OF THE INVENTION

A further problem with the device of US2020/000870 is that the ostium of the LAA has an irregular shape, which differs from patient to patient, resulting in imperfect contact between the inflatable balloon and the ostium. This is compounded by the presence of electrodes and arms on the inner surface of the balloon which decreases the compliancy of the balloon. The Applicant has addressed the problems of the prior art by providing a device with an energy delivery module that is proximal to the occlusive element and configured to be deployed into contact with the wall of the left atrium surrounding the ostium. The wall of the left atrium surrounding the ostium provides a more uniform surface allowing better contact between the energy delivery module and the tissue which results in better and more uniform tissue ablation around the ostium of the LAA. In one aspect, the energy delivery module comprises an array of arms configured for radial deployment into a radial array of arms suitable for contacting the wall of the left atrium surrounding the ostium of the LAA. This arrangement allows the arms to be deployed, and the energy delivery module axially adjusted into contact with the wall of the left atrium. The provision of through lumen in the radially expansible element also allows a sensor to be advanced into the LAA to detect whether the LAA has been successfully electrically isolated. In one embodiment, the sensor and energy delivery module both comprise electrodes allowing electrical signals to be passed between the electrodes across the tissue ablation zone to determine the level of electrical isolation.

In a first aspect, the invention provides a device for occlusion of a left atrial appendage of a heart, comprising:
an implantable occlusion apparatus comprising a radially expansible element that is adjustable between a contracted orientation suitable for transluminal delivery and a deployed orientation configured to occlude the left atrial appendage;
an elongated catheter member operably and detachably attached to the implantable occlusion apparatus and configured for transluminal delivery and deployment of the occlusion apparatus in the left atrial appendage; and
an energy delivery module configured for adjustment from a contracted configuration suitable for transluminal delivery and retraction, and a deployed configuration suitable for engagement with adjacent tissue to ablate the tissue,

wherein the energy delivery module is operably attached to the elongated catheter member proximally of the implantable occlusion apparatus and configured upon deployment to contact and ablate a wall of the left atrium surrounding an ostium of the left atrial appendage.

In any embodiment, the elongated catheter member and implantable occlusion apparatus when attached provide a through lumen configured to allow advancement of a sensor through the lumen into the left atrial appendage when the implantable occlusion apparatus is deployed in the left atrial appendage. This allows a sensor to be advanced through the catheter member into the LAA, and the measurement of electrical parameters of the tissue at the ablation zone between the sensor and energy delivery module while the LAA is actively occluded by the occlusion apparatus.

In any embodiment, the radially expansible occlusion apparatus comprises a cylindrical cage body having a sidewall, an optionally open distal end and a concave proximal end wall with a raised connecting hub with an open proximal end providing a through lumen into the cylindrical cage body, and a cover (which may be blood permeable or configured for in-vivo epithelialisation to make it blood impermeable) proximal of the raised connecting hub having a closable aperture providing access to the raised connecting hub from a proximal side of the occlusion apparatus.

In any embodiment, the energy delivery module comprises an electrode configured to ablate tissue optionally by ablating the tissue. The electrode may be configured for thermal or non-thermal tissue ablation.

In any embodiment, the electrode is disposed on a radially deployable member.

In any embodiment, the electrode is disposed at a tip or proximally of the tip of the radially deployable member.

In any embodiment, the radially deployable member comprises two spaced-apart electrodes. This arrangement allows tissue to be ablated at two ablation zones, and also allows electrical activity to be detected at two separate points. For example, the radially inner electrode may be employed to ablate tissue, and then the radially outward electrode can be employed as a sensing electrode to detect electrical activity between the radially outward electrode and a distant electrode (for example a sensing electrode disposed in the LAA) to determine electrical activity of the tissue between the electrodes and thereby determine if the tissue has been electrically isolated.

In any embodiment, one of the electrodes is disposed on or adjacent a tip and one of the electrodes is deployed proximally of the tip of the radially expansible member.

In any embodiment, the radially deployable member comprises one or more arms. The or each arm may be biased into a radially extended configuration. The or each arm may comprise one or more arm elements. The arms may be provided as a radial array typically configured to contact tissue of the left atrium around the ostium of the LAA. The or each arm may comprise a loop. The or each arm may comprise one or more electrodes. The or each arm may be resiliently deformable to allow the arm conform to a wall of the left atrium.

In any embodiment, the or each arm has a loop configuration comprising two loop elements, each of which when deployed radially contacts the wall of the left atrium.

In any embodiment, a distal tip of the loop comprises an electrode and at least one and preferably both of the loop elements include an electrode proximal of the tip of the loop.

In any embodiment, both loop elements include an plurality of electrodes proximal of the tip of the loop.

In any embodiment, the energy delivery module comprises an array of radially deployable members such as arms, for example 2, 3, 4, 5, or 6. In one embodiment, the energy delivery module comprises an array of loops.

In any embodiment, the or each radially deployable member is resiliently deformable to allow at least part of the radially deployable member conform the wall of the left atrium surrounding the ostium of the left atrial appendage.

In any embodiment, the radially deployable member comprises a first elongated control arm operably attached to proximal end of the radially deployable member and a second elongated control arm operably attached to distal end of the radially deployable member, whereby movement of the first control arm relative to the second control arm effects adjustment of the radially deployable member between the contracted and deployed configurations.

In any embodiment, the radially deployable member comprises an inflatable balloon that is typically configured to receive cryogenic liquid to cool the electrode during use.

In any embodiment, the energy delivery module is configured for rotational movement relative to the elongated catheter member. This allows the energy delivery module to be placed into a first tissue contact position, a first ablative treatment performed, and then rotated into a second position and a second ablative treatment performed. When the module comprises a number of arms with electrodes, this allows the re-positioning of the electrodes around the ostium of the LAA between each treatment providing improved circumferential ablation of the tissue.

In any embodiment, the energy delivery module is configured for axial movement relative to the implantable occlusion apparatus. This arrangement allows the position of the energy delivery module to be axially adjusted during positioning of the device. For example, the occlusion apparatus may be correctly positioned in the LAA first, and then the energy delivery module may be axially adjusted until it is in contact with the wall of the left atrium surrounding the ostium of the LAA.

In any embodiment, the elongated catheter member comprises a first catheter element operably and detachably attached to the implantable occlusion apparatus and a second catheter member operably attached to the energy delivery module. In any embodiment, the first and second catheter members are configured for relative axial and/or rotational movement. In any embodiment, the first catheter member is disposed inside a lumen of the second catheter member.

In another aspect, the invention provides a system comprising a device of the invention and an elongated deployment catheter for transluminal delivery of the device, in which a distal end of the elongated deployment catheter has a lumen for receipt of the device during transluminal delivery, and wherein the device and elongated deployment catheter are configured for relative axial movement to deploy the device proud of the distal end of the deployment catheter during use.

In any embodiment, the device is configured for adjustment between:
a first delivery configuration in which the implantable occlusion apparatus and energy delivery module are disposed within a distal end of the elongated deployment catheter;
a second partially deployed configuration in which the implantable occlusion apparatus is exposed distally of a distal end of the elongated deployment catheter and deployed and the energy delivery module is disposed within the distal end of the elongated deployment catheter;
a third fully deployed configuration in which the energy delivery module is exposed distally of a distal end of the elongated deployment catheter and deployed; and
a fourth configuration in which the elongated catheter member is detached from the implantable occlusion apparatus, the energy delivery module and the elongated catheter member are retracted into the elongated deployment catheter, and the elongated deployment catheter and energy delivery module are retracted.

In any embodiment, the energy delivery element comprises one or more electrodes, and the system comprises an electrical controller and processor operably connected to the or each electrode and actuable to energise the electrode(s) and/or receive electrical signals from the electrode(s) and/or send electrical signals to the electrode(s).

In any embodiment, the system comprises a sensor comprising an electrode configured for delivery through the lumen into the left atrial appendage, where the electrode is operatively connected to the electrical controller/processor.

In any embodiment, the processor is configured to detect an electrical parameter of a signal between the electrode of the energy delivery module and a sensor disposed in the left atrial appendage in contact with the wall of the left atrial appendage.

Thus, for example, the controller and processor may cause the electrode on the sensor in the LAA to emit a signal and the electrode of the energy delivery module to detect the signal (or vice versa) to determine if the tissue connecting the electrodes has been electrically isolated. For example, the voltage or impedance of the signal may be determined.

In any embodiment, the electrode of the radially deployable member is disposed at the tip of the radially deployable member.

In any embodiment, the energy delivery module comprises two electrodes, in which the processor is configured to detect an electrical parameter of a signal between the two electrodes.

In any embodiment, the electrical parameter is selected from voltage or electrical impedance.

In any embodiment, the electrical controller and processor are configured to electrically pace or electrically map the heart tissue.

In any embodiment, the energy delivery module is configured to cryogenically ablate tissue.

In any embodiment, the energy delivery module is a balloon configured to receive cryogenic liquid.

The invention also provides a method of occlusion and electrical isolation of the left atrial appendage that employs a device of the invention and comprises the steps of:
transluminally advancing the implantable occlusion apparatus attached to the delivery catheter until the implantable occlusion apparatus is disposed in the left atrial appendage (LAA);
radially deploying the implantable occlusion apparatus to anchor the apparatus in the LAA and occlude the LAA;
deploying the energy delivery module proximal of the implantable occlusion apparatus so that it is contact with target tissue in the left atrial wall surrounding an ostium of the LAA;
actuating the energy delivery module to ablate the target tissue;
optionally detecting if the LAA is electrically isolated;
detaching the delivery catheter from the connecting hub of the occlusion apparatus; and
withdrawing the delivery catheter and energy delivery module leaving the occlusion apparatus in-situ in the LAA.

In any embodiment, the method comprises a step of advancing a sensor through the elongated catheter member and implantable occlusion apparatus into the LAA and into contact with a wall of the LAA. The sensor may contact the wall of the LAA distal of the occlusion apparatus or through a wall of the occlusion apparatus.

In one embodiment, the step of detecting if the LAA is electrically isolated comprises sending an electrical signal between the sensor and an electrode forming part of the energy delivery module in contact with the wall of the left atrium. The detected signal may be correlated with electrical isolation of the tissue between the excitation and detection electrodes. The detected signal may comprise voltage or electrical impedance.

In any embodiment, the cover is configured to act as a scaffold for in-vivo endothelialisation, wherein the method includes the step of anchoring the device in the LAA and allowing in-vivo endothelialisation of the cover to occur.

In any embodiment, the occlusion apparatus is deployed first and the energy delivery module deployed thereafter.

In any embodiment, the energy delivery element is deployed first and the occlusion apparatus deployed thereafter.

In any embodiment, one of the occlusion apparatus and energy delivery element is first deployed, and the other of the occlusion apparatus and energy delivery element is adjusted axially and then deployed.

In any embodiment, the occlusion apparatus is first deployed, and then the energy delivery element is adjusted axially relative to the implanted occlusion apparatus into contact with the wall of the left atrium.

In a second aspect, the invention provides implantable radially expansible occlusion apparatus for occluding a body lumen such as a left atrial appendage of the heart and that is adjustable between a contracted orientation suitable for transluminal delivery and a deployed orientation configured to occlude the body lumen, the radially expansible occlusion apparatus comprising:

a cylindrical cage body having a sidewall, an optionally open distal end and a concave proximal end wall with a raised connecting hub with an open proximal end typically providing a through lumen into the cylindrical cage body; and a cover proximal of the raised connecting hub having a closable aperture providing access to the raised connecting hub from a proximal side of the occlusion apparatus.

The radially expansible occlusion apparatus typically comprises two wing elements mounted on opposed sides of the open proximal end of the raised connecting hub that are configured for movement from an at rest closed configuration in which the wing elements are folded over the open proximal end of the raised connecting hub to an open tensioned configuration. The wings are typically connected to the blood impermeable cover on each side of the aperture whereby when the wings are in the closed configuration the aperture in the blood impermeable cover is closed to prevent movement of blood through the raised connecting hub.

In any embodiment, the wings are formed from a shape memory material and are biased into the closed configuration.

In any embodiment, the cover comprises a first cover part attached to one wing and a second part attached to a second wing.

In any embodiment, the first and second cover parts are semi-circular.

In any embodiment, the two parts are configured to at least partially overlap when the wings are in the closed configuration.

In any embodiment, the cover is blood impermeable. In any embodiment, the cover is configured to act as a scaffold for in-vivo endothelialisation to make the cover blood impermeable.

In any embodiment, the cylindrical cage is formed from a wire mesh, typically a metal wire mesh, typically a wire mesh comprising a shape memory metal such as nitinol. The cylindrical cage may take other forms, for example a laser cut tube which may incorporate structural elements such as shape memory metal.

In any embodiment, the raised connecting is configured for detachable engagement with a distal end of an elongated catheter member.

In another aspect, the invention provides a body lumen occlusion system comprising an implantable radially expansible occlusion apparatus according to the invention, and an elongated catheter member, in which the raised connecting hub is configured for detachable engagement with a distal end of the elongated catheter member.

In a third aspect, the invention provides a device for occlusion of a left atrial appendage of a heart, comprising:
an implantable occlusion apparatus comprising a radially expansible element that is adjustable between a contracted orientation suitable for transluminal delivery and a deployed orientation configured to occlude the left atrial appendage, in which the radially expansible element comprises a proximal connecting hub configured for receipt of and electrical connection with an electrical supply module; and
an electrode module comprising an array of elongated electrode members terminating in and electrically connected to the proximal connecting hub, in which each electrode member in the array comprises at least one electrode and extends distally from the proximal connecting hub along at least a part of the side wall of the radially expansible element.

In any embodiment, each elongated electrode member has a proximal section that extends radially outwardly towards the sidewall of the radially expansible element and a distal section comprising the at least one electrode that extends axially and distally along at least part of the side wall of the radially expansible element.

In any embodiment, the distal section of each elongated electrode member comprises a plurality of spaced-apart electrodes.

In any embodiment, the radially expansible element comprises:

a cylindrical cage (for example a mesh cage) having a side wall, optionally an open distal end, and a concave proximal end wall, wherein the connecting hub is disposed on the concave proximal end wall and comprises an open proximal end typically providing a through lumen into the cylindrical cage body; and

a cover proximal of the connecting hub and having a closable aperture providing access to the connecting hub from a proximal side of the device.

In any embodiment, the electrode module is non-detachably attached to the side wall of the radially expansible element.

In any embodiment, the electrode module is integrated into the side wall of the mesh cage, or integrated into a membrane covering the cage.

In any embodiment, the electrode module is detachable from the radially expansible element, in which:

the side wall of the radially expansible element typically comprises an array of conduits dimensioned to receive at least the distal ends of the elongated electrode members; or in which the elongated electrode members are detachably attached to the side wall of the radially expansible element through frangible connections.

In any embodiment, the cover is blood impermeable. In any embodiment, the cover is configured to act as a scaffold for in-vivo endothelialisation to make the cover blood impermeable.

In any embodiment, the cylindrical cage is formed from a wire mesh, typically a metal wire mesh, typically a wire mesh comprising a shape memory metal such as nitinol. The cylindrical cage may take other forms, for example a laser cut tube which may incorporate structural elements such as shape memory metal.

In another aspect, the invention provides a system comprising a device of the invention and an electrical supply module configured for detachable attachment and electrical connection with the proximal connection hub.

In any embodiment, the electrical supply module is implantable and comprises a battery, in which the battery is optionally configured for remote charging.

In any embodiment, the electrical supply module comprises an elongated catheter member having a proximal end configured for electrical connection outside the body with an electrical supply.

In any embodiment, the system comprises a processor, in which the electrode module is configured to sense electrical activity of tissue of the body lumen via and transmit data relating to the electrical activity to the processor via the electrical supply module.

In a fourth aspect, the invention provides an implantable heart pacing system comprising:

an implantable radially expansible occlusion apparatus for occluding a left atrial appendage of the heart and that is adjustable between a contracted orientation suitable for transluminal delivery and a deployed orientation configured to occlude the left atrial appendage, the radially expansible occlusion apparatus comprising:
a cylindrical cage body having a sidewall, and a concave proximal end wall with a raised connecting hub having an open proximal end; and
a cover proximal of the raised connecting hub having a closable aperture providing access to the raised connecting hub from a proximal side of the occlusion apparatus, and

a heart chamber pacing device having a distal docking part configured for detachable engagement with the raised connecting hub and including a pulse generator, and a proximal part comprising at least one pacing lead electrically connected to the pulse generator and configured to contact a wall of a heart chamber and deliver electrical pulses to the wall of the heart.

In any embodiment, the heart chamber pacing device comprises a plurality of pacing leads, for example at least 2, 3, 4 or 5.

In any embodiment, one of the plurality of pacing leads is configured for pacing a first heart chamber, and in which a second of the plurality of pacing leads is configured for pacing a second heart chamber.

In any embodiment, the cover is blood impermeable. In any embodiment, the cover is configured to act as a scaffold for in-vivo endothelialisation to make the cover blood impermeable.

In any embodiment, the cylindrical cage is formed from a wire mesh, typically a metal wire mesh, typically a wire mesh comprising a shape memory metal such as nitinol. The cylindrical cage may take other forms, for example a laser cut tube which may incorporate structural elements such as shape memory metal.

In another aspect, the invention provides a method of pacing the heart with an implantable heart pacing apparatus according to the invention, comprising the steps of:
transluminally delivering the radially expansible occlusion apparatus into a left atrial appendage of the heart with a delivery catheter;
deploying the radially expansible occlusion apparatus in the left atrial appendage of the heart to (at least partially) occlude the left atrial appendage;
withdrawing the delivery catheter;
transluminally delivering the heart chamber pacing device into the left atrium of the heart with a delivery catheter and advancing the docking part through the blood impermeable cover of the radially expansible occlusion apparatus and into the raised connecting hub; and
withdrawing the delivery catheter to deploy the one or more pacing leads.

In any embodiment, the method comprises the steps of deploying one pacing leading into a first chamber of the heart and deploying a second pacing lead into a second chamber of the heart.

In a fifth aspect, the invention provides an implantable radially expansible occlusion apparatus for occluding a body lumen and that is adjustable between a contracted orientation suitable for transluminal delivery and a deployed orientation configured to occlude the body lumen, the radially expansible occlusion apparatus comprising:
a cylindrical cage body having a sidewall, a proximal end wall with a raised connecting hub having an open proximal end, and an annular shoulder section providing a transition between the proximal end wall and the side wall; and optionally, a cover proximal of the raised connecting hub having a closable aperture providing access to the raised connecting hub from a proximal side of the occlusion apparatus,

characterised in that the proximal end wall of the cylindrical cage is adjustable from a first configuration in which the annular shoulder section has a first diameter to a concave configuration in which the annular shoulder section has a second diameter greater than the first diameter, wherein the proximal end wall is configured for adjustment from the first configuration to the concave configuration by pushing raised connecting hub distally.

In any embodiment, the proximal end wall of the cylindrical cage is spring-adjustable from the first configuration to the concave configuration.

In any embodiment, the first configuration is convex (or non-inverted) and the second configuration is concave (or inverted).

In any embodiment, the cover is blood impermeable. In any embodiment, the cover is configured to act as a scaffold for in-vivo endothelialisation to make the cover blood impermeable.

In any embodiment, the cylindrical cage is formed from a wire mesh, typically a metal wire mesh, typically a wire mesh comprising a shape memory metal such as nitinol. The cylindrical cage may take other forms, for example a laser cut tube which may incorporate structural elements such as shape memory metal.

In another aspect, the invention provides a method of deploying an implantable radially expansible occlusion apparatus according to the invention, comprising the steps of:
transluminally delivering the radially expansible occlusion apparatus into a left atrial appendage of the heart with a delivery catheter having a distal end engaged with the raised connecting hub of the radially expansible occlusion apparatus;
deploying the radially expansible occlusion apparatus in the left atrial appendage of the heart with the proximal end wall of the cylindrical cage in a convex configuration such that the side wall of the cylindrical cage at least partially radially engages the wall of the left atrial appendage; and
advancing the delivery catheter to push the raised connecting hub distally to force the proximal end wall into the concave configuration to fully radially engage the wall of the left atrial appendage.

In any embodiment, the method comprises a step of determining the level of radial engagement between the proximal end wall and the wall of the left atrial appendage prior to changing the configuration of the proximal end wall.

In a sixth aspect, the invention provides an implantable heart parameter sensing system comprising:

an implantable radially expansible occlusion apparatus for occluding a left atrial appendage of the heart and that is adjustable between a contracted orientation suitable for transluminal delivery and a deployed orientation configured to occlude the left atrial appendage, the radially expansible occlusion apparatus comprising:
a cylindrical cage body having a sidewall, an optionally open distal end, and a concave proximal end wall with a raised connecting hub with an open proximal end;
a cover proximal of the raised connecting hub having a closable aperture providing access to the raised connecting hub from a proximal side of the occlusion apparatus,
a sensing module having a docking part disposed within the raised connecting hub and a sensing part configured to detect at least one parameter of the heart; and
a wireless communication module disposed within the cylindrical cage body and in electrical communication with the sensing module and configured to wirelessly transmit sensed heart parameter data to a processor located outside of the body.

In any embodiment, the system comprises software for a computing device configured to cause the computing device communicate with and receive sensed heart parameter data from the wireless communications module, and optionally display the data on a screen of the computing device.

In any embodiment, the software is downloadable software and in the which the computing device is a mobile communications device.

In any embodiment, the software is configured to cause the computing device to electronically transmit the received heart parameter data to another computing device.

In any embodiment, the wireless communications module comprises a coil configured for deployment in the left atrial appendage distally of the cylindrical cage body.

In any embodiment, the sensing module is configured to detect at least one parameter of the heart selected from:
atrial, ventricular and pulmonary pressure changes;
atrial fibrillation detection;
electrical Changes such as voltage ECG etc;
pH Changes;
hemodynamic flow changes;
hematological changes; and
respiratory rate.

In any embodiment, the sensing part of the sensing module is configured to extend from the docking part at least partly in the left atrial appendage distal of the cylindrical cage body and/or from the docking part at least partly in a chamber of the heart and for example transeptally into a right side of the heart.

In any embodiment, the cover is blood impermeable. In any embodiment, the cover is configured to act as a scaffold for in-vivo endothelialisation to make the cover blood impermeable.

In any embodiment, the cylindrical cage is formed from a wire mesh, typically a metal wire mesh, typically a wire mesh comprising a shape memory metal such as nitinol. The cylindrical cage may take other forms, for example a laser cut tube which may incorporate structural elements such as shape memory metal.

In a seventh aspect, the invention provides a system comprising:

an implantable radially expansible occlusion apparatus for occluding a body lumen and that is adjustable between a contracted orientation suitable for transluminal delivery and a deployed orientation configured to occlude the body lumen, the radially expansible occlusion apparatus comprising:
a cylindrical cage body having a sidewall, a proximal end wall with a raised connecting hub having an open proximal end, and an optionally open distal end; and
a cover proximal of the raised connecting hub having a closable aperture providing access to the raised connecting hub from a proximal side of the occlusion apparatus, and

a delivery catheter for delivering the implantable radially expansible occlusion apparatus to a body lumen to be occluded.

Typically, a distal part of the delivery catheter comprises a collet lock mechanism. The collet lock mechanism typically comprises:
a sleeve member coupled to the delivery catheter and configured for slidable movement relative to the delivery catheter into the raised connecting hub, the sleeve member comprising a distal end adjustable from a radially narrowed configuration (for example a radially inwardly tapered configuration) to a radially widened configuration (typically a radially outwardly tapered configuration), in which the distal end is biased into the radially narrowed configuration; and
a mandrel configured for slidable movement into the sleeve member and urging of the distal end of the sleeve member into the radially widened configuration to lock the sleeve member and delivery catheter to the implantable radially expansible occlusion apparatus.

In any embodiment, the distal end of the sleeve member comprises radially outward flanges configured to abut a distal end of the connection hub when the sleeve member is urged into the radially widened configuration.

In any embodiment, the distal end of the sleeve member comprises arms that are adjustable from a radially inwardly tapered configuration to a radially outwardly tapered configuration.

The devices and methods of the invention may be employed to narrow, fully or partially occlude, or fully or partially devascularise a body lumen.

The devices and methods of the invention may be employed to prevent or treat a condition of the heart, for example:

treatment or prevention of an arrhythmia or atrial fibrillation, prevention of a thrombotic event,

or treatment or prevention of ischaemia or a hypertensive disorder, in a subject.

In one embodiment, the subject has a LAA and the method is a method of preventing or treating atrial fibrillation in the subject by occluding and electrically isolating the LAA.

In one embodiment, the body lumen is a heart valve opening, for example the aortic valve opening, and wherein the method is a method of narrowing the (aortic) valve opening, for example prior to (aortic) valve replacement. The invention also relates to a method of (aortic) valve replacement comprising an initial step of narrowing the (aortic) valve opening by a method of the invention, or by using a device or system of the invention. Thus, the method of the invention may be employed to narrow the aortic valve opening prior to trans aortic valve implantation.

Other aspects and preferred embodiments of the invention are defined and described in the other claims set out below.

### BRIEF DESCRIPTION OF THE FIGURES

FIG. 1 (comparative) illustrates a radially expansible element of the prior art.

FIG. 2 (comparative) illustrates the radially expansible element of FIG. A with a cover on a proximal end thereof distal of the connecting hub.

FIG. 3A is a sectional side elevation view illustrating an occlusion apparatus according to a first aspect of the invention in a partially deployed configuration.

FIG. 3B is a sectional side elevation view illustrating the occlusion apparatus of FIG. 1A showing the energy delivery module partially deployed.

FIG. 3C is a sectional side elevation view illustrating the occlusion apparatus of FIG. 1A in situ in the heart showing the energy delivery module fully deployed and in contact with a wall of the LAA around the LAA ostium.

FIG. 3D is a side elevational view of the energy delivery module is partial deployment.

FIG. 3E is an end view of the energy delivery module is a fully deployed configuration.

FIG. 3F shows the radially expansible element in-situ in the LAA after the catheter and energy delivery module have been detached and retracted.

FIG. 4A is a side elevational view illustrating an occlusion apparatus according to a first aspect of the invention in a partially deployed configuration.

FIG. 4B is a side elevational view illustrating the occlusion apparatus of FIG. 1A showing the radially expansible element fully exposed proud of the delivery catheter.

FIG. 4C is a side elevational view illustrating the occlusion apparatus showing the energy delivery module fully exposed and prior to deployment.

FIG. 4D is a sectional side elevation view illustrating the occlusion apparatus of showing the energy delivery module fully deployed.

FIG. 5A is a section side elevational view of part of a proximal concave surface of a radially expansible member according to the invention showing wing elements in an open configuration.

FIG. 5B is a section side elevational view of the proximal concave surface of a radially expansible member of FIG. 5A showing the wing elements in a folded closed configuration.

FIG. 6A is an elevational view of an occlusion apparatus according to the invention with a proximal part of the cage cut-away to illustrate an electrode array.

FIG. 6B is a detailed view of the connecting hub of the occlusion apparatus and an electrical supply module.

FIG. 7 is an illustration of a device for pacing the heart shown implanted in a left atrial appendage of the heart with a pacing lead extending proximally into the left ventricle.

FIG. 8A is a side sectional view of an occlusion apparatus of the invention implanted in the left atrial appendage of the heart prior to adjustment of the apparatus to expand the proximal end of the device.

FIG. 8B is a side sectional view of an occlusion apparatus of FIG. 8A after adjustment of the apparatus to expand the proximal end of the device and shoulder the device into the wall of the left atrial appendage.

FIG. 8C shows the occlusion apparatus in an expanded configuration of FIG. 8B and a closure device (inset) having a mesh cap at a proximal end and a distal end comprising a hub and arms. The arms and hub are advanced through the hub of the occlusion apparatus and the mesh cap fits over the proximal end of the device and the arms function to torque the mesh cap distally.

FIG. 9A is a side sectional view of an occlusion apparatus of the invention having a wireless communications coil.

FIG. 9B is an illustration of how the device of FIG. 9A communicates with a local mobile device and communicates onward with remote devices.

FIGS. 10A to 10E illustrate an occlusion apparatus and delivery catheter with collet-type locking mechanism for releasably attaching the catheter to the occlusion apparatus.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

“Implantable occlusion apparatus” means an apparatus configured for implantation in a body lumen, especially implantation in the heart at least partially within the left atrial appendage, and upon actuation to at least partially occlude the body lumen resulting in partial or complete devascularisation of the body lumen. The occlusion apparatus is detachably connected to a delivery catheter which delivers the occlusion apparatus to the target site, and typically remains attached during occlusion, sensing and energy delivery treatments and is generally detached after the energy delivery treatment and removed from the body leaving the occlusion apparatus (or the radially expansible element part of the occlusion apparatus) implanted in the body lumen. Occlusion may be complete occlusion (closing) of the body lumen or partial occlusion (narrowing of the body lumen or near complete occlusion). The occlusion apparatus comprises a body that is expansible from a contracted delivery configuration to an expanded deployed configuration. The body may take many forms, for example a wireframe structure formed from a braided or meshed material. Examples of expandable wireframe structures suitable for transluminal delivery are known in the literature and described in, for example, WO01/87168, U.S. Pat. No. 6,652,548, US2004/219028, U.S. Pat. Nos. 6,454,775, 4,909,789, 5,573,530, WO2013/109756. Other forms of bodies suitable for use with the present invention include plate or saucer shaped scaffolds, or inflatable balloons, or stents. In one embodiment, the body is formed from a metal, for example a shape-memory metal such as nitinol. The body may have any shape suitable for the purpose of the invention, for example cylindrical, discoid or spheroid. In one preferred embodiment, the apparatus comprises a cylindrical body, for example a cylindrical cage body. In one embodiment, the body comprises a tissue ablation device. In one embodiment, the ablation device comprises an array of electrical components. In one embodiment, the array of electrical components are configured to deliver ablative energy in a specific pattern while mapping temperature. In one embodiment, the array of electrical components are configured for pacing the cardiac tissue for confirmation of ablation and disruption of chaotic signalling from the LAA. In one embodiment, a distal face of the radially expansible body comprises a covering configured to promote epithelial cell proliferation. In one embodiment, the body comprises a stepped radial force stiffness profile from distal to proximal device. In one embodiment, the body comprises a metal mesh cage scaffold. In one embodiment, a coupling between the body and the catheter member is located distally to the left atrial facing side of the body. In one embodiment, the body in a deployed configuration has a radial diameter at least 10% greater than the radial diameter of the left atrial appendage at a point of deployment. In one embodiment, the furthermost distal body is configured to be atraumatic to cardiac tissue. In one embodiment, the body covering is configured to self-close on retraction of the delivery component (i.e. catheter member). In one embodiment, the body comprises a braided mesh scaffold that in one embodiment is conducive to collagen infiltration on thermal energy delivery to promote increased anti migration resistance. In one embodiment, the array of electrodes generate an electrical map or profile of the ablation zone and the surrounding tissue electrical impedance measurements to characterise the electrical properties of the tissue, wherein the characterisation is optionally used as a measurement and confirmation of ablation effectiveness.

“Body lumen” means a cavity in the body, and may be an elongated cavity such as a vessel (i.e. an artery, vein, lymph vessel, urethra, ureter, sinus, auditory canal, nasal cavity, bronchus) or an annular space in the heart such as the left atrial appendage, left ventricular outflow tract, the aortic valve, the mitral valve, mitral valve continuity, or heart valve or valve opening.

“Detachably attached” means that the device is configured such that the occlusion apparatus is attached to the elongated delivery catheter during delivery and can be released after deployment and treatment whereby the occlusion apparatus, or just the radially expansible element part of the occlusion apparatus, is implanted in the heart and the elongated delivery catheter can be withdrawn leaving the occlusion apparatus (or the radially expansible element) in-situ. Typically, the device includes a control mechanism for remotely detaching the occlusion apparatus or radially expansible element from the elongated catheter member. Typically, an actuation switch for the control mechanism is disposed on the control handle.

“Transluminal delivery” means delivery of the occlusion apparatus to a target site (for example the heart) heart through a body lumen, for example delivery through an artery or vein. In one embodiment, the device of the invention is advanced through an artery or vein to deliver the occlusion apparatus to the left atrium of the heart and at least partially in the LAA. In one embodiment, the device is delivered such that the distal body is disposed within the LAA and the proximal body is disposed in the left atrium just outside the LAA. In one embodiment, the device is delivered such that the distal body is disposed within the LAA and the proximal body is disposed in the left atrium abutting a mouth of the LAA. In one embodiment, the device is delivered such that both the distal body and proximal body are disposed within the LAA.

“Cover” typically means a layer covering the proximal side of radially expansible element proximal of the connecting hub. It may be formed from a woven mesh material, and may include a re-closable aperture, for example an overlapping flap of material. It may be configured to prevent blood flow past the occlusion apparatus into the LAA, or configured to act as a scaffold for in-vivo endothelialisation.

“Covering/cover configured to act as a scaffold for in-vivo endothelialisation” means a material that is use promotes epithelialisation of the distal or proximal body. In one embodiment, the covering is a membrane that comprises agents that promote epithelial cell proliferation. Examples include growth factors such as fibroblast growth factor, transforming growth factor, epidermal growth factor and platelet derived growth factor, cells such as endothelial cells or endothelial progenitor cells, and biological material such as tissue or tissue components. Examples of tissue components include endothelial tissue, extracellular matrix, sub-mucosa, dura mater, pericardium, endocardium, serosa, peritoneum, and basement membrane tissue. In one embodiment, the covering is porous. In one embodiment, the covering is a biocompatible scaffold formed from biological material. In one embodiment, the covering is a porous scaffold formed from a biological material such as collagen. In one embodiment, the covering is a lyophilised scaffold.

“Sensor” means an electrical sensor configured to detect an environmental parameter within or proximal of the LAA, for example blood flow, electrical signal activity, pressure, impedance, moisture or the like. The sensor may include an emission sensor and a detection sensor that are suitably spaced apart. In one embodiment, the sensor is an electrode. In one embodiment, the sensor is configured to detect fluid flow. In one embodiment, the sensor is configured to detect electrical conductivity. In one embodiment, the sensor is configured to detect electrical impedance. In one embodiment, the sensor is configured to detect an acoustic signal. In one embodiment, the sensor is configured to detect an optical signal typically indicative of changes in blood flow in the surrounding tissue. In one embodiment, the sensor is configured to detect stretch. In one embodiment, the sensor is configured to detect moisture. In one embodiment, the sensor is configured for wireless transmission of a detected signal to a processor. The sensor may be employed in real time during the method of the invention to allow a surgeon determine when the LAA is sufficiently occluded, for example determining blood flow or electrical activity within the LAA. Examples suitable sensor include optical sensors, radio frequency sensors, microwave sensors, sensors based on lower frequency electromagnetic waves (i.e. from DC to RF), radiofrequency waves (from RF to MW) and microwave sensors (GHz). In one embodiment, the device of the invention is configured for axial movement of the sensor relative to the radially expansible body. In one embodiment, sensor comprises a radially expansible body. In one embodiment, the device of the invention is configured for rotational movement of the sensor, typically about a longitudinal axis of the device or an axis co-parallel with a longitudinal axis of the device. This helps positioning of the sensor, and helps achieve full circumferential tissue ablation.

“Optical sensor” means a sensor suitable for detecting changes in blood flow in tissue, and which generally involves directing light at the tissue and measuring reflected/transmitted light. These sensors are particularly sensitive for detecting changes in blood flow in adjacent tissue, and therefore suitable for detecting devascularisation of tissue such as the LAA. Examples include optical probes using pulse oximetry, photoplasmography, near-infrared spectroscopy, Contrast enhanced ultrasonography, diffuse correlation spectroscopy (DCS), transmittance or reflectance sensors, LED RGB, laser doppler flowometry, diffuse reflectance, fluorescence/autofluoresence, Near Infrared (NI R) imaging, diffuse correlation spectroscopy, and optical coherence tomography. An example of a photopeasmography sensor is a device that passes two wavelengths of light through the tissue to a photodetector which measures the changing absorbance at each of the wavelengths, allowing it to determine the absorbances due to the pulsing arterial blood alone, excluding venous blood, muscle, fat etc). Photoplesmography measures change in volume of a tissue caused by a heartbeat which is detected by illuminating the tissue with the light from a single LED and then measuring the amount of light either reflected to a photodiode.

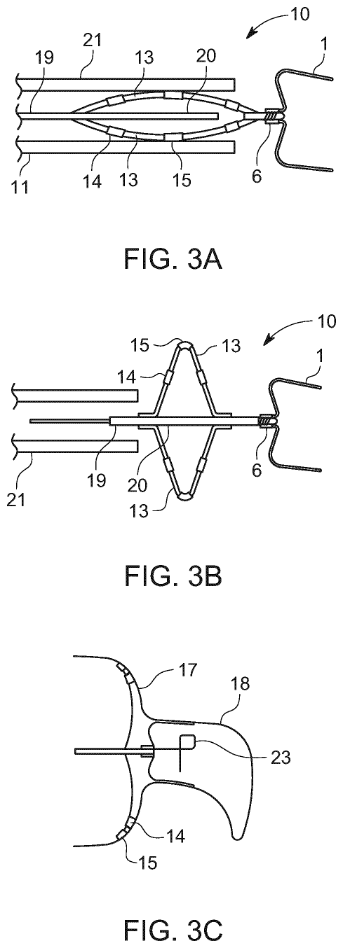

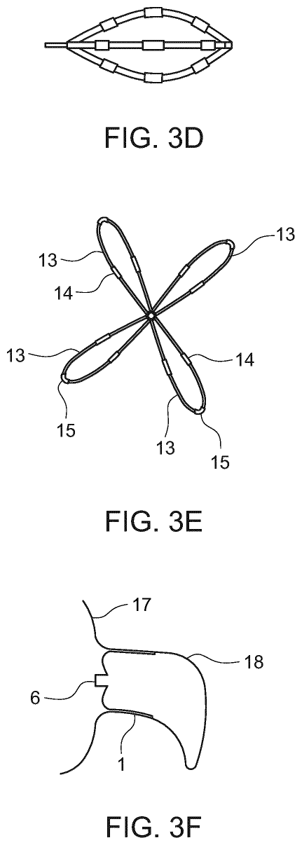

“Energy delivering element” or “energy delivery module” refers to a device configured to receive energy and direct the energy to the tissue, and in one embodiment convert the energy to heat to heat the tissue causing collagen denaturation (tissue ablation). The energy delivery element may be thermal (e.g. RF ablation probe) or non-thermal (pulse field ablation probe). Tissue ablating energy delivery modules are known to the skilled person, and operate on the basis of emitting thermal energy (heat or cold), microwave energy, radiofrequency energy, electroporation energy, other types of energy suitable for ablation of tissue, or chemicals configured to ablate tissue. Tissue ablation devices are sold by ANGIODYNAMICS, including the STARBURST radiofrequency ablation systems, and ACCULIS microwave ABLATION SYSTEMS. Examples of tissue ablating chemicals include alcohol, heated saline, heated water. Typically, the liquid is heated to at least 45° C., ie 45-60° C. In one embodiment, the tissue ablation device comprises an array of electrodes or electrical components typically configured to deliver heat to adjacent tissue. (alcohol, heated saline, heated water) In one embodiment, one or more of the electrodes comprises at least one or two thermocouples in electrical communication with the electrode. In one embodiment, one or more of the electrodes are configured to deliver RF or microwave energy. In one embodiment, the device of the invention is configured for axial movement of the energy delivery element relative to the radially expansible body. In one embodiment, energy delivery element comprises a radially expansible body. In one embodiment, the device of the invention is configured for rotational movement of the energy delivery element, typically about a longitudinal axis of the device or an axis co-parallel with a longitudinal axis of the device. This helps positioning of the energy delivering element, and helps achieve full circumferential tissue ablation. In one embodiment, the energy delivery module comprises one or more arms. Typically, the energy delivery module comprises a plurality of arms, typically configured upon deployment in a radial array (examples of radial arrays of arms are illustrated in FIGS. 3C and 3E). The arms are typically deployable from an axial orientation to a radially deployed orientation, typically extending radially outwardly from a central axis of the device. The arms may be biased into the radially deployed orientation, and may be deployed by exposing the arms proud of a distal end of a delivery catheter. The module may comprise, for example, 2, 4, 6 or 8 arms. Each arm may be in the form of a loop (e.g. a wire loop) having two loop elements/arms and a distal apex. The arm generally includes an electrode, and may include a plurality of electrodes. In one embodiment, the arm has an outer electrode and an inner electrode disposed radially inwardly of the outer electrode. The outer electrode may be disposed at or adjacent to a distal end of the arm. When the arm is a loop, each loop element/arm and the distal apex may include electrodes. When the module comprises an array of radially extending arms each having outer and inner electrodes, the outer electrodes may define an outer circumferential tissue treatment/sensing zone and the inner electrodes may define an inner circumferential tissue treatment/sensing zone. In a preferred embodiment, the outer electrodes are configured for sending a tissue parameter and the inner electrodes are configured for tissue ablation.

“Atrial fibrillation” or “AF” is a common cardiac rhythm disorder affecting an estimated 6 million patients in the United States alone. AF is the second leading cause of stroke in the United States and may account for nearly one-third of strokes in the elderly. In greater than 90% of cases where a blood clot (thrombus) is found in the AF patient, the clot develops in the left atrial appendage (LAA) of the heart. The irregular heart beat in AF causes blood to pool in the left atrial appendage, because clotting occurs when blood is stagnant, clots or thrombi may form in the LAA. These blood clots may dislodge from the left atrial appendage and may enter the cranial circulation causing a stroke, the coronary circulation causing a myocardial infarction, the peripheral circulation causing limb ischemia, as well as other vascular beds. The term includes all forms of atrial fibrillation, including paroxysmal (intermittent) AF and persistent and longstanding persistent AF (PLPAF).

“Ischaemic event” refers to a restriction in blood supply to a body organ or tissue, resulting in a shortage of oxygen and glucose supply to the affected organ or tissue. The term includes stroke, a blockage of blood supply to a part of the brain caused by a blood clot blocking the blood supply to the brain and the resultant damage to the affected part of the brain, and transient ischaemic events (TIA's), also known as “mini-strokes”, which are similar to strokes but are transient in nature and generally do not cause lasting damage to the brain. When the restriction in blood supply occurs in the coronary arteries, the ischaemic event is known as a myocardial infarction (MI) or heart attack.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

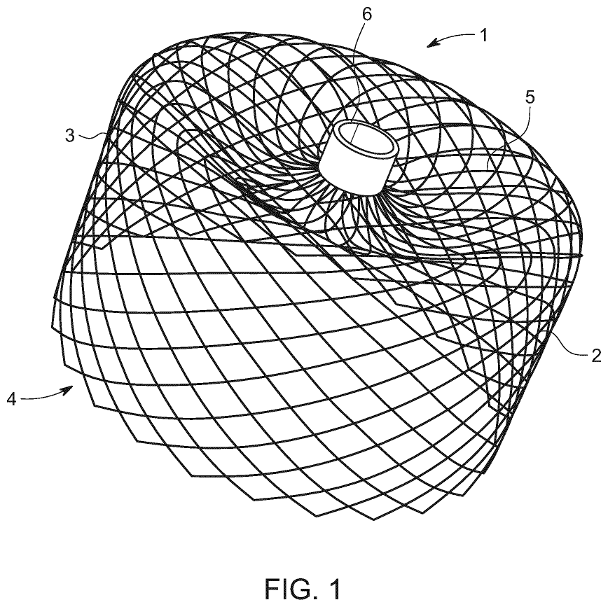

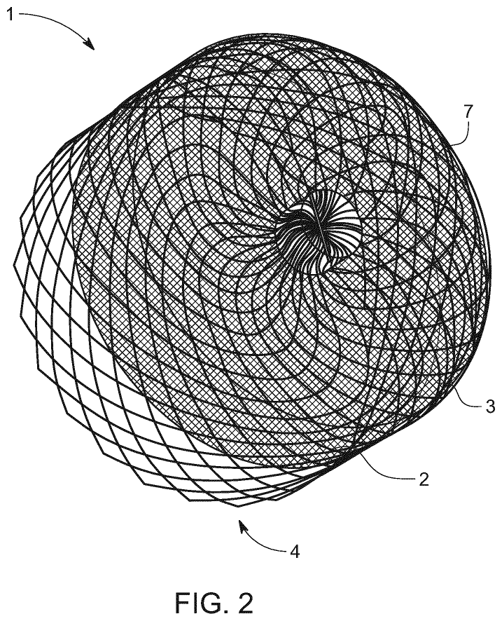

FIG. 1 (comparative) illustrates a radially expansible element of the prior art 1 comprising a cylindrical nitinol mesh cage 2 with a sidewall 3, open distal end 4 and concave proximal end wall 5 with a raised connection hub 6. FIG. 2 (comparative) illustrates the radially expansible element 1 with a blood impermeable cover 7 on a proximal end thereof distal of the connecting hub 6 and including a re-closable aperture 8.

FIGS. 3A to 3F illustrate a device for occlusion of a left atrial appendage, indicated generally by the reference numeral 10, in which parts identified with reference to previous embodiments are assigned the same reference numerals. In this embodiment, a delivery catheter 11 is detachably attached to the connecting hub 6 of radially expansible element 1 for transluminal delivery of the radially expansible element to the left atrial appendage of the heart. The device 10 comprises an energy delivery module 12 attached to the delivery catheter proximal of the cage 2, and comprising four loop elements 13 configured for movement upon deployment from a radially contracted orientation shown in FIG. 3A to a partially radially extended orientation shown in FIG. 3B and fully radially extended orientation shown in elevational view in FIG. 3C and end view in FIG. 3E. Each loop element 13 comprises two arms each having an inner electrode 14 and an apex/tip having an outer electrode 15. The electrodes 14 and 15 are independently controllable by a controller (not shown) and configured to ablate tissue and send or receive electrical signals and communicate data to a processor (not shown).

As illustrated in FIG. 3C, when fully deployed, the loop elements 13 are radially arranged about the central axis of the delivery catheter, and configured to face the wall of the left atrium 17 surrounding the ostium of the left atrial appendage 18 with both electrodes 14 and 15 in contact with the tissue. The delivery catheter 11 comprises an outer sheath 21 that is configured for axial proximal movement relative to the energy delivery module and cage 2 to deploy the cage and the energy delivery module, and control arms 19 and 20 for deployment and retraction of the loop elements 13 of the energy delivery module. It will be appreciated that the loop elements may be configured for self-expansion once the outer sheath 21 has been retracted. The inner control arm also includes a lumen allowing a sensing or treatment catheter be passed through the lumen and connecting hub into the left atrial appendage. The delivery catheter maybe detached from the connecting hub and withdrawn along with the energy delivery module leaving the radially expansible element 1 in-situ occluding the left atrial appendage of the heart.

In use, the device is delivered transluminal to the left atrium of the heart by the delivery catheter with the radially expansible element and energy delivery module in a contracted configuration and stowed in the distal end of the delivery catheter. Once in the left atrium, the catheter is advanced so that the tip is within the LAA, and the outer sheath 21 is retracted to deploy the radially expansible element 1 in the LAA with the walls of the element in circumferential contact with a wall of the LAA. The outer sheath 21 is then further retracted exposing the loop elements 13 of the energy delivery module, and the control arms are actuated to deploy the loop elements 13 into a radially expanded orientation. The catheter may be adjusted axially to ensure that the loop elements are in contact with the tissue of the left atrium. Once in position, a controller is actuated to energise the electrodes to electrically ablate tissue surrounding the ostium of the LAA. During treatment, the energy delivery module may be rotated to ensure full circumferential ablation of tissue around the ostium of the LAA.

Once treatment has been completed, a controller and a processor may be used to determine the level of electrical isolation of the tissue by monitoring electrical signals between spaced apart electrodes. This may be achieved in a number of ways: for example, electrodes in contact with the wall of the left atrium on opposed sides of the LAA may be employed as excitation and detection electrodes to determine the electrical conductivity of the tissue between the electrodes (the detected signal may be voltage or electrical impedance). In another embodiment, an electrode 15 in contact with the wall of the left atrium and an electrode 23 in contact with the wall of the LAA may be used as excitation and detection electrodes. The electrode in the LAA may be part of the device, or a separate sensor which is transluminally advanced into the LAA through the delivery catheter and connection hub. In one preferred embodiment, tissue ablation is performed by electrodes distal of the tip of the loop elements and subsequently the detection step employs the electrode on the tip of the loop element and an electrode in the LAA, thereby detecting electrical conductivity across the ablated tissue zone.

If the detection step determines that the treated tissue has not bee sufficiently ablated (i.e. the LAA is not electrically isolated), a further treatment step can be performed. Once it has been determined that the LAA is electrically isolated, the loop elements are radially retracted and withdrawn into the delivery catheter, the catheter is detached from the connection hub 6, and transluminal retracted leaving the radially expansible element in-situ.

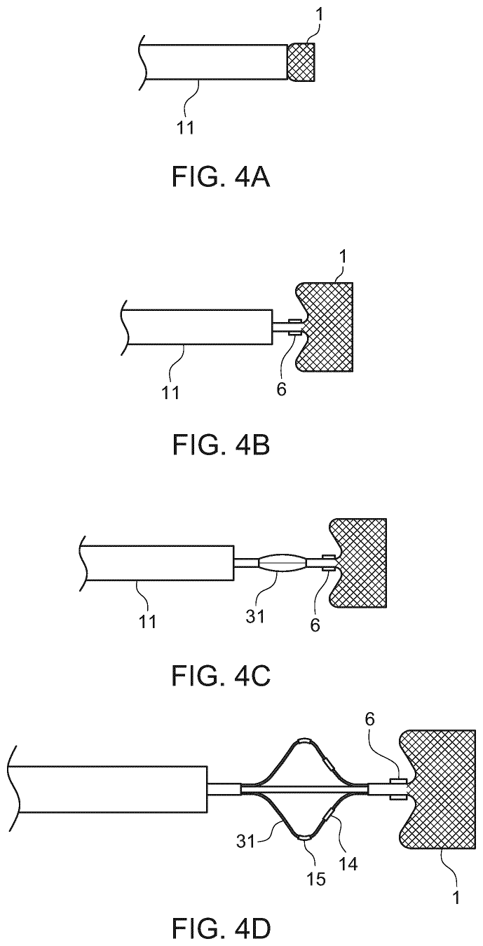

Referring to FIGS. 4A to 4D, an alternative embodiment of a device of the invention is described, indicated generally by the reference numeral 30, in which parts described with reference to the previous embodiment is assigned the same reference numerals. In this embodiment, the energy delivery module is a balloon 31 mounted to the delivery catheter 11 and fluidically connected to a cryogenic fluid vessel (not shown) via a lumen in the delivery catheter that is configured to supply cryogenic fluid to the balloon. The balloon 31 comprises electrodes 14 and 15 mounted on a surface of the balloon, that are connected to an electrical controller and processor as described previously. The use of this device is substantially the same as that described with reference to the previous embodiment, whereby the radially expansible element 1 is first deployed and anchored in the LAA 18 before the balloon 31 is exposed in the left atrium 17 adjacent the ostium of the LAA and inflated into contact with the wall of the left atrium surrounding the ostium of the LAA. The treatment and detection steps are as described previously.

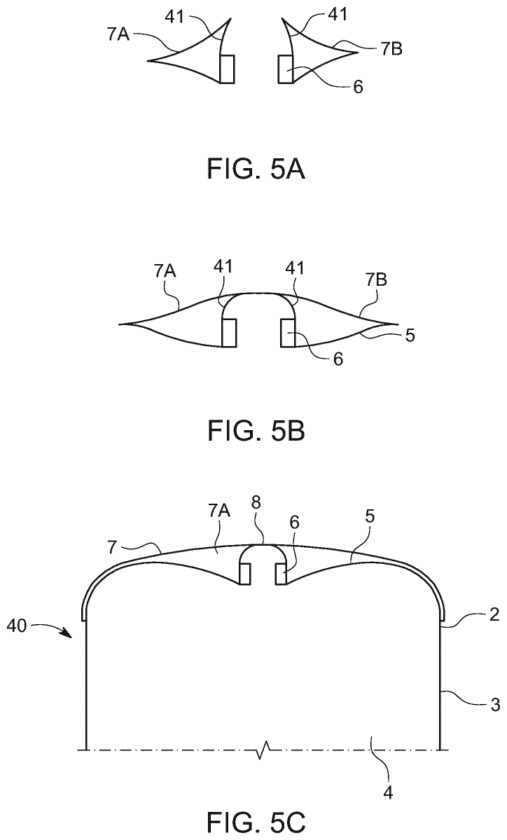

Referring to FIGS. 5A to 5C, an implantable radially expansible occlusion apparatus 40 for occluding a body lumen such as a left atrial appendage of the heart is described. The apparatus is radially adjustable between a contracted orientation suitable for transluminal delivery and a deployed orientation configured to occlude the body lumen, and comprises a cylindrical cage body 2 having a sidewall 3, an open distal end 4 and a concave proximal end wall 5 with a raised connecting hub 6 with an open proximal end providing a through lumen into the cylindrical cage body. A blood impermeable cover 7 is mounted to the proximal end wall 5 proximal of the raised connecting hub 5 and comprises a re-closable aperture 8 providing access to the raised connecting hub from a proximal side of the occlusion apparatus.

The radially expansible occlusion apparatus 40 comprises two wing elements 41 mounted on opposed sides of the open proximal end of the raised connecting hub 6 that are configured for movement from an at rest closed configuration in which the wing elements are folded over the open proximal end of the raised connecting hub (FIG. 5B) to an open tensioned configuration (FIG. 5A), and wherein the wings are connected to the blood impermeable cover 7 on each side of the aperture 8 whereby when the wings are in the closed configuration the aperture in the blood impermeable cover is closed to prevent movement of blood through the raised connecting hub. The wings 41 are formed from a shape memory material and are biased into a curved closed configuration shown in FIG. 5B. In the embodiment shown the cover 7 comprises a first semi-circular cover part 7A attached to one wing and a second semi-circular part 7B attached to a second wing.

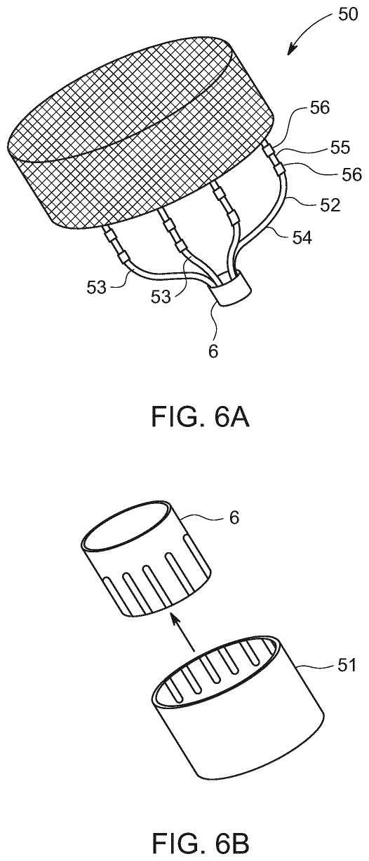

Referring to FIGS. 6A and 6B, there is illustrated a device for occlusion of a left atrial appendage of a heart, indicated generally by the reference numeral 50. The device comprises a radially expansible element as described in the previous embodiments in which parts described previously are assigned the same reference numerals. In this embodiment, the proximal connecting hub 6 is configured for receipt of and electrical connection with an electrical supply module 51 which may be an implantable element configured for detachable engagement with the hub 6 (as shown in FIG. 6B) and having a battery, or may be a catheter member having a distal end configured for electrical connection with the hub 6 and a proximal end connected outside the body to an electrical supply.

The device 50 comprises an electrode module 52 comprising an array of elongated electrode members 53 terminating in and electrically connected to the proximal connecting hub 6, in which each electrode member in the array comprises a proximal section 54 that extends radially outwardly from the proximal connecting hub 6 towards the sidewall 3 of the radially expansible element and a distal section 55 that extends distally along at least a part of the side wall 3 of the radially expansible element 1 and having at least one electrode 56. In this embodiment, the electrode module 52 is non-detachably attached to the side wall 3 (or a membrane attached to the side wall) of the radially expansible element, and remains in-situ in the LAA attached to the LAA after a tissue ablation treatment has been performed. The electrode members may be disposed inside cage or outside the cage or may weave in and out of the cage mesh.

In another embodiment, the electrode module 52 may be detachable from the radially expansible element, and the side wall 3 of the radially expansible element may comprise an array of conduits dimensioned to receive at least the distal ends of the elongated electrode members. Alternatively, the elongated electrode members may be detachably attached to the side wall of the radially expansible element through frangible connections to allow for retraction of the electrode members after a tissue ablation treatment.

In one embodiment, the system may include a processor and controller operatively connected to the electrode module, in which the electrode module is configured to sense electrical activity of tissue of the body lumen via and transmit data relating to the electrical activity to the processor via the electrical supply module.

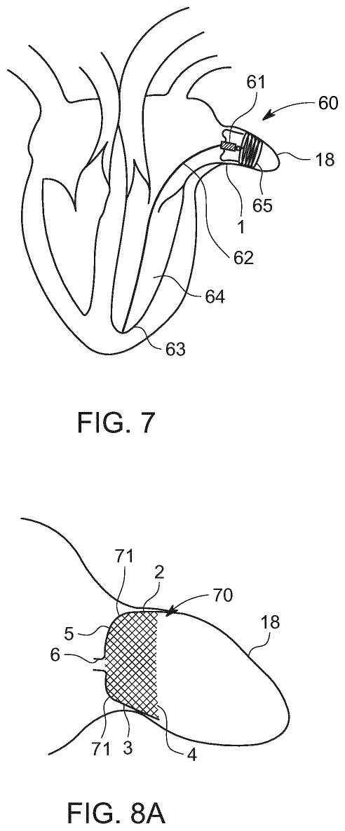

Referring to FIG. 7, a heart pacing device of the invention is described, indicated generally by the reference numeral 60, in which parts described with reference to the previous embodiments are assigned the same reference numerals. The heart pacing device comprises a radially expansible element 1 as described previously, and a heart chamber pacing device having a distal docking part 61 configured for detachable engagement with the raised connecting hub 6 and including a pulse generator, and a proximal part comprising at least one pacing lead 62 electrically connected to the pulse generator and configured to contact a wall 63 of a heart chamber (in the embodiment shown, the left ventricle 64) and deliver electrical pulses to the wall of the heart. The device comprises a communications coil 65. Although not illustrated, the device may include a plurality of pacing leads, for example at least 2, 3, 4 or 5 and one of the plurality of pacing leads may be configured for pacing a first heart chamber, and a second of the plurality of pacing leads may be configured for pacing a second heart chamber.

A method use of the device comprises transluminally delivering the radially expansible occlusion apparatus into a left atrial appendage of the heart with a delivery catheter, deploying the radially expansible occlusion apparatus in the left atrial appendage of the heart to occlude the left atrial appendage, withdrawing the delivery catheter, transluminally delivering the heart chamber pacing device into the left atrium of the heart with a delivery catheter and advancing the docking part through the blood impermeable cover of the radially expansible occlusion apparatus and into the raised connecting hub, withdrawing the delivery catheter to deploy the one or more pacing leads.

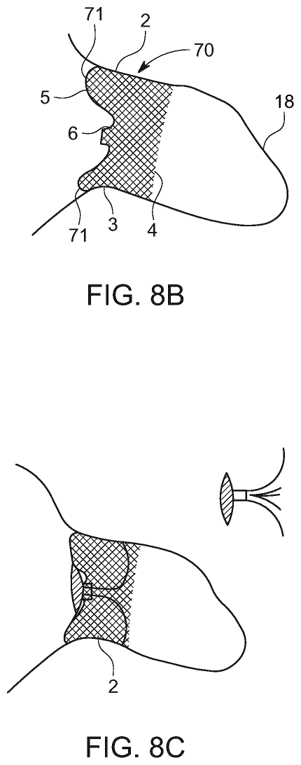

Referring to FIGS. 8A and 8B, an implantable radially expansible occlusion apparatus for occluding a body lumen, indicated generally by the reference numeral 70 is described, and in which parts described with reference to the previous embodiments are assigned the same reference numerals. This embodiment provides a radially expansible element that can be adjusted while anchored in LAA to shoulder the device more fully into contact with the wall of the LAA. The radially expansible occlusion apparatus 70 comprising a cylindrical cage body 2 having a sidewall 3, a proximal end wall 5 with a raised connecting hub 6 having an open proximal end, an annular shoulder section 71 providing a transition between the proximal end wall 5 and the side wall 2, and a blood impermeable cover 7. The proximal end wall 5 of the cylindrical cage is spring-adjustable from a first convex configuration in which the annular shoulder section has a first diameter (FIG. 6A) to a concave configuration in which the annular shoulder section has a second diameter greater than the first diameter (FIG. 8B). The proximal end wall is configured for adjustment from the first configuration to the concave configuration by pushing raised connecting hub 6 distally, which may be achieved by the advancing the delivery catheter while it is connected to the connecting hub 6. FIG. 8C shows a closure device (inset) having a mesh cap at a proximal end and a distal end comprising a hub and arms. The arms and hub are advanced through the hub 6 of the occlusion apparatus and the mesh cap fits over the proximal end of the device and the arms function to torque the mesh cap distally.

A method of use of this device comprises transluminally delivering the radially expansible occlusion apparatus into a left atrial appendage of the heart with a delivery catheter having a distal end engaged with the raised connecting hub of the radially expansible occlusion apparatus, deploying the radially expansible occlusion apparatus in the left atrial appendage of the heart with the proximal end wall of the cylindrical cage in a convex configuration such that the side wall of the cylindrical cage at least partially radially engages the wall of the left atrial appendage, and advancing the delivery catheter to push the raised connecting hub distally to force the proximal end wall into the concave configuration to fully radially engage the wall of the left atrial appendage.

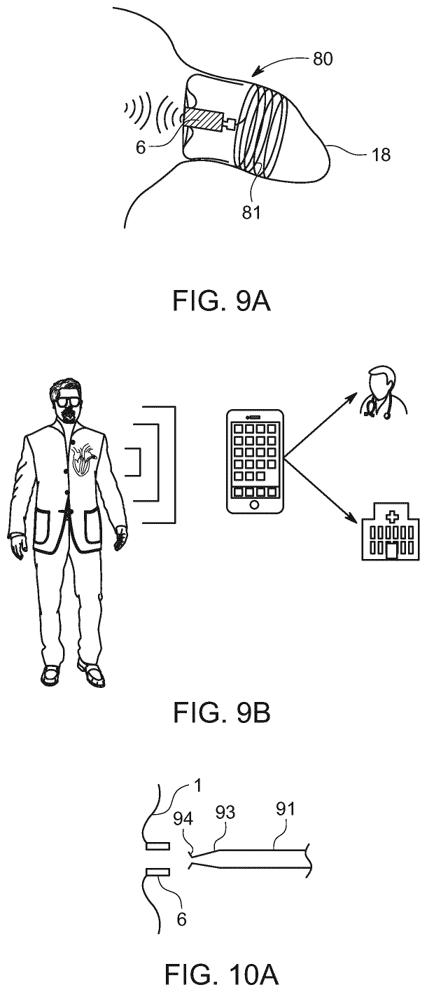

Referring to FIG. 9A, an implantable heart parameter sensing system, indicated generally by the reference numeral 80, is described in which parts described with reference to previous embodiments are assigned the same reference numerals. In this embodiment, the system 80 comprises a radially expansible occlusion apparatus 1 as previously described. The system comprises a sensing module having a docking part (not shown) disposed within the raised connecting hub 6 and a sensing part (not shown) configured to detect at least one parameter of the heart, and a wireless communication coil 81 disposed within the cylindrical cage body and in electrical communication with the sensing module and configured to wirelessly transmit sensed heart parameter data to a processor located outside of the body. The sensing module is configured to detect at least one parameter of the heart selected from atrial, ventricular and pulmonary pressure changes, atrial fibrillation detection, electrical Changes such as voltage ECG etc, pH Changes, hemodynamic flow changes, hematological changes, and respiratory rate. The system includes software for a computing device configured to cause the computing device communicate with and receive sensed heart parameter data from the wireless communications module, and display the data on a screen of the computing device or electronically transmit the received heart parameter data to another computing device (as illustrated in FIG. 9B). The software may be downloadable software for a mobile communications device such as a Tablet or a smart phone. Although not shown the sensing part of the sensing module may configured to extend from the docking part at least partly in the left atrial appendage distal of the cylindrical cage body and/or from the docking part at least partly in a chamber of the heart and for example transeptally into a right side of the heart.

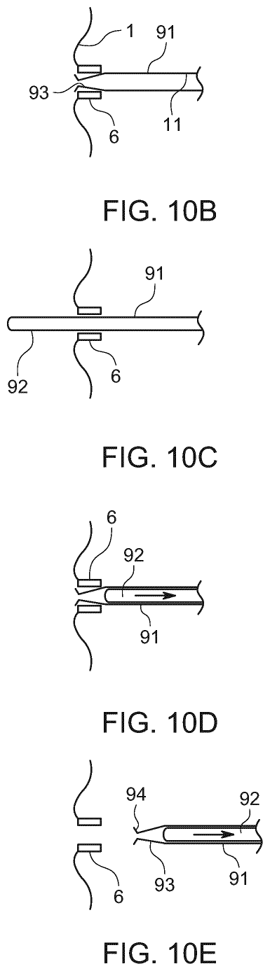

Referring to FIGS. 10A to 10E, there is illustrated a body lumen occlusion apparatus according to the invention, indicated generally by the reference numeral 90, in which parts described with reference to previous embodiments are assigned the same reference numerals. This invention is characterised by a delivery catheter 11 having collet locking mechanism to lock the delivery catheter to the connection hub 6 of the cage 2. The collet locking mechanism comprises a sleeve member 91 and cooperating mandrel 92. The sleeve member 91 is coupled to a distal end of the delivery catheter 11 and configured for slidable movement relative to the delivery catheter into the raised connecting hub 6, and comprises a distal end 93 adjustable from a relaxed radially inwardly tapered configuration (FIGS. 10A and 10B) to a radially outwardly tapered configuration (FIG. 10C). The mandrel 92 is configured for slidable movement into the sleeve member 91 to urge the distal end of the sleeve member into the outwardly tapered configuration to lock the sleeve member and delivery catheter to the implantable radially expansible occlusion apparatus. The distal end 93 of the sleeve member comprises radially outward flanges 94 configured to abut a distal end of the connection hub 6 when the sleeve member is urged into the radially outwardly tapered configuration to lock the delivery catheter to the connection hub (FIG. 10C), Removal of the mandrel 92 allows the distal end of the sleeve member 91 to return to a relaxed inwardly tapered configuration (FIG. 10D), releasing the sleeve member and allowing retracting of the delivery catheter (FIG. 10E).

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A device for occlusion of a left atrial appendage of a heart, comprising:
an implantable occlusion apparatus comprising a radially expansible element that is adjustable between a contracted orientation suitable for transluminal delivery and a deployed orientation configured to occlude the left atrial appendage;
an elongated catheter member operably and detachably attached to the implantable occlusion apparatus and configured for transluminal delivery and deployment of the occlusion apparatus in the left atrial appendage; and
an energy delivery module configured for adjustment from a contracted configuration suitable for transluminal delivery and retraction, and a deployed configuration suitable for engagement with adjacent tissue to ablate the tissue,
wherein the energy delivery module is operably attached to the elongated catheter member proximally of the radially expansible element and configured upon deployment to contact and ablate a section of a wall of the left atrium surrounding an ostium of the left atrial appendage.

2. A device according to claim 1, in which the elongated catheter member and implantable occlusion apparatus when attached provide a through lumen configured to allow advancement of a sensor through the through lumen into the left atrial appendage when the implantable occlusion apparatus is deployed in the left atrial appendage.

3. A device according to claim 1, in which the energy delivery module comprises an array of radially deployable arms, each arm comprising an electrode configured to ablate tissue.

4. A device according to claim 1, in which the energy delivery module comprises an array of radially deployable arms, each arm comprising an electrode configured to ablate tissue, and in which the electrodes are disposed at or adjacent a tip of the radially deployable arms.

5. A device according to claim 14 in which the energy delivery module comprises an array of radially deployable arms, each arm comprising an electrode configured to ablate tissue, and in which the electrodes are disposed at or adjacent a tip of the radially deployable arms, and including an outer electrode disposed at or adjacent to the tip of the radially deployable arm and an inner electrode disposed radially inwardly of the outer electrode.

6. A device according to claim 3, in which at least one of the radially deployable arm has a loop configuration comprising two loop elements, configured to radially contact the wall of the left atrium around the ostium of the left atrial appendage when deployed.

7. A device according to claim 6, in which a distal tip of the loop comprises an outer electrode and one or both of the loop elements include an inner electrode proximal of the tip of the loop.

8. A device according to claim 3 in which the radially deployable arms are resiliently deformable to allow at least part of the radially deployable arm to conform to the wall of the left atrium surrounding the ostium of the left atrial appendage.

9. A device according to claim 1, in which the radially deployable member comprises an inflatable balloon, in which the inflatable balloon is configured to receive cryogenic liquid.

10. A device according to claim 1, in which the energy delivery module is configured for rotational movement relative to the elongated catheter member.

11. A device according to claim 1, in which the energy delivery module is configured for axial movement relative to the implantable occlusion apparatus.

12. A device according to claim 1, including an outer deployment catheter that is axially adjustable relative to the elongated catheter member, in which the device is configured for adjustment between:
a first delivery configuration in which the implantable occlusion apparatus and energy delivery module are disposed within a distal end of the elongated deployment catheter;
a second partially deployed configuration in which the implantable occlusion apparatus is exposed distally of a distal end of the elongated deployment catheter and deployed and the energy delivery module is disposed within the distal end of the elongated deployment catheter;
a third fully deployed configuration in which the energy delivery module is exposed distally of a distal end of the elongated deployment catheter and deployed; and
a fourth configuration in which the elongated catheter member is detached from the implantable occlusion apparatus, the energy delivery module and the elongated catheter member are retracted into the elongated deployment catheter, and the elongated deployment catheter and energy delivery module are retracted.

13. A system comprising a device according to claim 1 and an electrical controller operably connected to a processor, in which the energy delivery module comprises an array of radially deployable arms, each arm comprising an electrode configured to ablate tissue, and in which the electrical controller is operably connected to the electrode and actuable to energize the electrode and/or receive electrical signals from the electrode and/or send electrical signals to the electrode.

14. A system according to claim 13, in which the processor is configured to detect an electrical parameter of a signal between the electrode of the energy delivery module and a sensing electrode disposed in the left atrial appendage in contact with the wall of the left atrial appendage.

15. A system according to claim 13, in which the processor is configured to detect an electrical parameter of a signal between the electrode of the energy delivery module and a sensing electrode disposed in the left atrial appendage in contact with the wall of the left atrial appendage, the system including a sensor comprising an electrode configured for delivery through the through lumen into the left atrial appendage, wherein the processor is configured to detect an electrical parameter of the signal between the electrode of the radially deployable member and the electrode of the sensor disposed in the left atrial appendage to determine electrical isolation of the left atrial appendage.

16. A device according to claim 13, in which the electrical controller and processor are configured to pass an electrical signal between two electrodes and detect an electrical parameter of a signal between the two electrodes.
